SEQUENCE LISTING (25 sequences)

SEQ ID NO: 1 - DNA, 117 bp, Burkholderia glumae
atggtcagatcgatgcgttccagggtggcggcgagggcggtggcatgggcgttggcggtgatgccgctggccggcgcggccgggttgacgatggccgcgtcgcccgcggccgtcgcg

SEQ ID NO: 2 - protein, 39 aa, Burkholderia glumae
MVRSMRSRVAARAVAWALAVMPLAGAAGLTMAASPAAVA

SEQ ID NO: 3 - DNA, 244 bp, Burkholderia glumae
gatcgatcgcgcccgcttgcgcgccgcagcatccgcgccgtcatatgtccacccgccgcgcgcgcgcggcgctgtccatcgagtagagacgcctatccaaacggccgtctgattgcagacaggagccgcgccgccatgtttcactccgcacttgccgctcgagcgtgcccgacgacctgagaacggcgcggcgccgcgcggcgtggcattccgatcgacgtaaccgataacgatggagataaac

SEQ ID NO: 4 - DNA, 361 bp, Burkholderia glumae
gatcgatcgcgcccgcttgcgcgccgcagcatccgcgccgtcatatgtccacccgccgcgcgcgcgcggcgctgtccatcgagtagagacgcctatccaaacggccgtctgattgtagacaggagccgcgccgccatgtttcactccgcacttgccgctcgagcgtgcccgacgacctgagaacggcgcggcgccgcgcggcgtggcattccgatcgacgtaaccgataacgatggagataaacatggtcagattgatgcgttccagggtggcggcgagggcggtggcatgggcgttggcggtgatgccgctggccggcgcggccgggttgacgatggccgcgtcgcccgcggccgtcgcg

SEQ ID NO: 5 - DNA, 2905 bp, Burkholderia glumae
ggatcctgccttgagcccgggcgcgtcccggacgcgcttccgggttcatccccgacccgttctgaattcaccttgaacgcaggcgtttcgcgcgcggcgcggccttcgcgctgcgccgcaatacgtctcgcgccgtgtcatgtcgattcgcgatgcaatcgtcggcaatcggcgtgattgttgcgcccgcaacctgatcgccgcccgcgcccgcgtggcgcgcgcgcggcacgccattcaccggatcgatcgcgcccgcttgcgcgccgcagcatccgcgccgtcatatgtccacccgccgcgcgcgcgcggcgctgtccatcgagtagagacgcctatccaaacggccgtctgattgtagacaggagccgcgccgccatgtttcactccgcacttgccgctcgagcgtgcccgacgacctgagaacggcgcggcgccgcgcggcgtggcattccgatcgacgtaaccgataacgatggagataaacatggtcagattgatgcgttccagggtggcggcgagggcggtggcatgggcgttggcggtgatgccgctggccggcgcggccgggttgacgatggccgcgtcgcccgcggccgtcgcggcggacacctacgcggcgacgcgctatccggtgatcctcgtccacggcctcgcgggcaccgacaagttcgcgaacgtggtggactattggtacggaatccagagcgatctgcaatcgcatggcgcgaaggtgtacgtcgcgaatctctcgggattccagagcgacgacgggccgaacggccgcggcgagcagctgctcgcctacgtgaagcaggtgctcgcggccaccggcgcgaccaaggtgaacctgatcggccacagccagggcggcctgacctcgcgctacgtcgcggccgtcgcgccgcaactggtggcctcggtgacgacgatcggcacgccgcatcgcggctccgagttcgccgacttcgtgcaggacgtgctgaagaccgatccgaccgggctctcgtcgacggtgatcgccgccttcgtcaacgtgttcggcacgctcgtcagcagctcgcacaacaccgaccaggacgcgctcgcggcgctgcgcacgctcaccaccgcgcagaccgccacctacaaccggaacttcccgagcgcgggcctgggcgcgcccggttcgtgccagacgggcgccgcgaccgaaaccgtcggcggcagccagcacctgctctattcgtggggcggcaccgcgatccagcccacctccaccgtgctcggcgtgaccggcgcgaccgacaccagcaccggcacgctcgacgtcgcgaacgtgaccgacccgtccacgctcgcgctgctcgccaccggcgcggtgatgatcaatcgcgcctcggggcagaacgacgggctcgtctcgcgctgcagctcgctgttcgggcaggtgatcagcaccagctaccactggaaccatctcgacgagatcaaccagctgctcggcgtgcgcggcgccaacgcggaagatccggtcgcggtgatccgcacgcacgtgaaccggctcaagctgcagggcgtgtgatggcgcaggccgatcgtccggcgcgcggcgggctggccgcgcgcccgatgcgcggcgcgtcgttcgcgctggccgggctcgtcgcgtgtgccgcctgtgccgcggtcgtgctgtggcttcggcccgccgccccgtcgcccgcgccggccggcgccgtcgcgggcgggccggcggccggcgtgcccgccgcggcaagcggcgcggcggaggccgccatgccgttgccggcggcgctgccgggcgcgctggctggctcgcatgcgccgcgcctgccgctggccgccggcggccggctcgcgaggacgcgcgcggtgcgcgagttcttcgactattgcctgaccgcgcagggcgaactgacgcccgccgcgctcgatgcgctggtgcggcgcgagatcgccgcgcagcttgacggcagccccgcgcaagcggaggcgctcggcgtctggcgccgctatcgcgcctacttcgacgcgctcgcgcaattgcccggcgacggcgcggtgctcggcgacaagctcgatccggccgccatgcagctcgcgctcgatcagcgcgcggcgctggccgaccgcacgctcggcgagtgggccgagccgttcttcggcgacgagcagcgccggcagcgccatgacctcgaacggatccggatcgccaacgacaccacgctgagccctgagcagaaggccgcgcggcttgccgcgctcgacgcgcagctgacgccggacgagcgcgcgcagcaggcggcgctgcatgcgcagcaggacgcggtgacgaagatcgccgacttgcagaaggccggcgcgacgcccgaccagatgcgcgcgcagatcgcgcagacgctcgggcccgaggcggccgcgcgcgccgcgcagatgcagcaggacgacgaggcgtggcagacgcgctatcaagcctatgcggccgagcgcgaccggatcgcggcgcaggggctcgcgccgcaggatcgcgatgcgcggatcgcgcagctcaggcagcagactttcacggcgccgggggaggcgatccgcgcggcgtcgctcgatcgcggcgcgggcggttagggggcgccggcgtgccgggcaccgtgtgcttcgcgagtgcttcgaacgggtggggccgcacggcgtttccagccgctgcatcgcgtgtttcgtactgaaatggcatgagtgacagcgtgccgacagcgtgctgacagggttcccggtttttgcctttccacgtgctttcattggcgccgcgagcgagcaagaatcacgacgctctgcaacaatgcggggcgatggcgcgtttgacggtcggaatcgatgcaaacgcgccgccgcgtcgttccatccgcgg

SEQ ID NO: 6 - protein, 319 aa, Burkholderia glumae
ADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV

SEQ ID NO: 7 - protein, 353 aa, Burkholderia glumae
MAQADRPARGGLAARPMRGASFALAGLVACAACAAVVLWLRPAAPSPAPAGAVAGGPAAGVPAAASGAAEAAMPLPAALPGALAGSHAPRLPLAAGGRLARTRAVREFFDYCLTAQGELTPAALDALVRREIAAQLDGSPAQAEALGVWRRYRAYFDALAQLPGDGAVLGDKLDPAAMQLALDQRAALADRTLGEWAEPFFGDEQRRQRHDLERIRIANDTTLSPEQKAARLAALDAQLTPDERAQQAALHAQQDAVTKIADLQKAGATPDQMRAQIAQTLGPEAAARAAQMQQDDEAWQTRYQAYAAERDRIAAQGLAPQDRDARIAQLRQQTFTAPGEAIRAASLDRGAGG

SEQ ID NO: 8 - DNA, 117 bp, Burkholderia glumae
atggtcagattgatgcgttccagggtggcggcgagggcggtggcatgggcgttggcggtgatgccgctggccggcgcggccgggttgacgatggccgcgtcgcccgcggccgtcgcg

SEQ ID NO: 9 - protein, 39 aa, Burkholderia glumae
MVRLMRSRVAARAVAWALAVMPLAGAAGLTMAASPAAVA

SEQ ID NO: 10 - DNA, 244 bp, Burkholderia glumae
gatcgatcgcgcccgcttgcgcgccgcagcatccgcgccgtcatatgtccacccgccgcgcgcgcgcggcgctgtccatcgagtagagacgcctatccaaacggccgtctgattgtagacaggagccgcgccgccatgtttcactccgcacttgccgctcgagcgtgcccgacgacctgagaacggcgcggcgccgcgcggcgtggcattccgatcgacgtaaccgataacgatggagataaac

SEQ ID NO: 11 - DNA, 2662 bp, Burkholderia glumae
gatcgatcgcgcccgcttgcgcgccgcagcatccgcgccgtcatatgtccacccgccgcgcgcgcgcggcgctgtccatcgagtagagacgcctatccaaacggccgtctgattgtagacaggagccgcgccgccatgtttcactccgcacttgccgctcgagcgtgcccgacgacctgagaacggcgcggcgccgcgcggcgtggcattccgatcgacgtaaccgataacgatggagataaacatggtcagattgatgcgttccagggtggcggcgagggcggtggcatgggcgttggcggtgatgccgctggccggcgcggccgggttgacgatggccgcgtcgcccgcggccgtcgcggcggacacctacgcggcgacgcgctatccggtgatcctcgtccacggcctcgcgggcaccgacaagttcgcgaacgtggtggactattggtacggaatccagagcgatctgcaatcgcatggcgcgaaggtgtacgtcgcgaatctctcgggattccagagcgacgacgggccgaacggccgcggcgagcagctgctcgcctacgtgaagcaggtgctcgcggccaccggcgcgaccaaggtgaacctgatcggccacagccagggcggcctgacctcgcgctacgtcgcggccgtcgcgccgcaactggtggcctcggtgacgacgatcggcacgccgcatcgcggctccgagttcgccgacttcgtgcaggacgtgctgaagaccgatccgaccgggctctcgtcgacggtgatcgccgccttcgtcaacgtgttcggcacgctcgtcagcagctcgcacaacaccgaccaggacgcgctcgcggcgctgcgcacgctcaccaccgcgcagaccgccacctacaaccggaacttcccgagcgcgggcctgggcgcgcccggttcgtgccagacgggcgccgcgaccgaaaccgtcggcggcagccagcacctgctctattcgtggggcggcaccgcgatccagcccacctccaccgtgctcggcgtgaccggcgcgaccgacaccagcaccggcacgctcgacgtcgcgaacgtgaccgacccgtccacgctcgcgctgctcgccaccggcgcggtgatgatcaatcgcgcctcggggcagaacgacgggctcgtctcgcgctgcagctcgctgttcgggcaggtgatcagcaccagctaccactggaaccatctcgacgagatcaaccagctgctcggcgtgcgcggcgccaacgcggaagatccggtcgcggtgatccgcacgcacgtgaaccggctcaagctgcagggcgtgtgatggcgcaggccgatcgtccggcgcgcggcgggctggccgcgcgcccgatgcgcggcgcgtcgttcgcgctggccgggctcgtcgcgtgtgccgcctgtgccgcggtcgtgctgtggcttcggcccgccgccccgtcgcccgcgccggccggcgccgtcgcgggcgggccggcggccggcgtgcccgccgcggcaagcggcgcggcggaggccgccatgccgttgccggcggcgctgccgggcgcgctggctggctcgcatgcgccgcgcctgccgctggccgccggcggccggctcgcgaggacgcgcgcggtgcgcgagttcttcgactattgcctgaccgcgcagggcgaactgacgcccgccgcgctcgatgcgctggtgcggcgcgagatcgccgcgcagcttgacggcagccccgcgcaagcggaggcgctcggcgtctggcgccgctatcgcgcctacttcgacgcgctcgcgcaattgcccggcgacggcgcggtgctcggcgacaagctcgatccggccgccatgcagctcgcgctcgatcagcgcgcggcgctggccgaccgcacgctcggcgagtgggccgagccgttcttcggcgacgagcagcgccggcagcgccatgacctcgaacggatccggatcgccaacgacaccacgctgagccctgagcagaaggccgcgcggcttgccgcgctcgacgcgcagctgacgccggacgagcgcgcgcagcaggcggcgctgcatgcgcagcaggacgcggtgacgaagatcgccgacttgcagaaggccggcgcgacgcccgaccagatgcgcgcgcagatcgcgcagacgctcgggcccgaggcggccgcgcgcgccgcgcagatgcagcaggacgacgaggcgtggcagacgcgctatcaagcctatgcggccgagcgcgaccggatcgcggcgcaggggctcgcgccgcaggatcgcgatgcgcggatcgcgcagctcaggcagcagactttcacggcgccgggggaggcgatccgcgcggcgtcgctcgatcgcggcgcgggcggttagggggcgccggcgtgccgggcaccgtgtgcttcgcgagtgcttcgaacgggtggggccgcacggcgtttccagccgctgcatcgcgtgtttcgtactgaaatggcatgagtgacagcgtgccgacagcgtgctgacagggttcccggtttttgcctttccacgtgctttcattggcgccgcgagcgagcaagaatcacgacgctctgcaacaatgcggggcgatggcgcgtttgacggtcggaatcgatgcaaacgcgccgccgcgtcgttccatccgcgg

SEQ ID NO: 12 - DNA, 960 bp, Burkholderia glumae
gcggacacctacgcggcgacgcgctatccggtgatcctcgtccacggcctcgcgggcaccgacaagttcgcgaacgtggtggactattggtacggaatccagagcgatctgcaatcgcatggcgcgaaggtgtacgtcgcgaatctctcgggattccagagcgacgacgggccgaacggccgcggcgagcagctgctcgcctacgtgaagcaggtgctcgcggccaccggcgcgaccaaggtgaacctgatcggccacagccagggcggcctgacctcgcgctacgtcgcggccgtcgcgccgcaactggtggcctcggtgacgacgatcggcacgccgcatcgcggctccgagttcgccgacttcgtgcaggacgtgctgaagaccgatccgaccgggctctcgtcgacggtgatcgccgccttcgtcaacgtgttcggcacgctcgtcagcagctcgcacaacaccgaccaggacgcgctcgcggcgctgcgcacgctcaccaccgcgcagaccgccacctacaaccggaacttcccgagcgcgggcctgggcgcgcccggttcgtgccagacgggcgccgcgaccgaaaccgtcggcggcagccagcacctgctctattcgtggggcggcaccgcgatccagcccacctccaccgtgctcggcgtgaccggcgcgaccgacaccagcaccggcacgctcgacgtcgcgaacgtgaccgacccgtccacgctcgcgctgctcgccaccggcgcggtgatgatcaatcgcgcctcggggcagaacgacgggctcgtctcgcgctgcagctcgctgttcgggcaggtgatcagcaccagctaccactggaaccatctcgacgagatcaaccagctgctcggcgtgcgcggcgccaacgcggaagatccggtcgcggtgatccgcacgcacgtgaaccggctcaagctgcagggcgtgtga

SEQ ID NO: 13 - DNA, 1062 bp, Burkholderia glumae
atggcgcaggccgatcgtccggcgcgcggcgggctggccgcgcgcccgatgcgcggcgcgtcgttcgcgctggccgggctcgtcgcgtgtgccgcctgtgccgcggtcgtgctgtggcttcggcccgccgccccgtcgcccgcgccggccggcgccgtcgcgggcgggccggcggccggcgtgcccgccgcggcaagcggcgcggcggaggccgccatgccgttgccggcggcgctgccgggcgcgctggctggctcgcatgcgccgcgcctgccgctggccgccggcggccggctcgcgaggacgcgcgcggtgcgcgagttcttcgactattgcctgaccgcgcagggcgaactgacgcccgccgcgctcgatgcgctggtgcggcgcgagatcgccgcgcagcttgacggcagccccgcgcaagcggaggcgctcggcgtctggcgccgctatcgcgcctacttcgacgcgctcgcgcaattgcccggcgacggcgcggtgctcggcgacaagctcgatccggccgccatgcagctcgcgctcgatcagcgcgcggcgctggccgaccgcacgctcggcgagtgggccgagccgttcttcggcgacgagcagcgccggcagcgccatgacctcgaacggatccggatcgccaacgacaccacgctgagccctgagcagaaggccgcgcggcttgccgcgctcgacgcgcagctgacgccggacgagcgcgcgcagcaggcggcgctgcatgcgcagcaggacgcggtgacgaagatcgccgacttgcagaaggccggcgcgacgcccgaccagatgcgcgcgcagatcgcgcagacgctcgggcccgaggcggccgcgcgcgccgcgcagatgcagcaggacgacgaggcgtggcagacgcgctatcaagcctatgcggccgagcgcgaccggatcgcggcgcaggggctcgcgccgcaggatcgcgatgcgcggatcgcgcagctcaggcagcagactttcacggcgccgggggaggcgatccgcgcggcgtcgctcgatcgcggcgcgggcggttag

SEQ ID NO: 14 - DNA, 30 bp, artificial
  primer sequence
atatatatctagaattcaccggatcgatcg

SEQ ID NO: 15 - DNA, 28 bp, artificial
  primer sequence
atatataagcttacccgttcgaagcact

SEQ ID NO: 16 - DNA, 22 bp, artificial
  primer sequence
cctgtctacaatcagacggccg

SEQ ID NO: 17 - DNA, 22 bp, artificial
  primer sequence
cggccgtctgattgtagacagg

SEQ ID NO: 18 - DNA, 22 bp, artificial
  primer sequence
ggaacgcatcaatctgaccatg

SEQ ID NO: 19 - DNA, 22 bp, artificial
  primer
catggtcagattgatgcgttcc

SEQ ID NO: 20 - DNA, 19 bp, artificial
  primer sequence
ctatccggtgatcctcgtc

SEQ ID NO: 21 - DNA, 20 bp, artificial
  primer sequence
gagagattcgcgacgtacac

SEQ ID NO: 22 - DNA, 20 bp, artificial
  primer sequence
gtggcagacgcgctatcaag

SEQ ID NO: 23 - DNA, 21 bp, artificial
  primer sequence
cgtgaaagtctgctgcctgag

SEQ ID NO: 24 - DNA, 20 bp, artificial
  primer sequence
gatgacgacgcaacccagag

SEQ ID NO: 25 - DNA, 20 bp, artificial
  primer sequence
gaacgcttccttcagcagca

(19) United States
(12) Patent Application Publication
(10) Pub. No.: US 2016/0298095 A1
(43) Pub. Date: Oct. 13, 2016

(54) Nucleic acid molecules for increased protein production

(71) Applicants: Andreas Knapp, Duesseldorf (DE); Sonja Voget, Goettingen (DE); Rolf Daniel, Goettingen (DE); Karl-Erich Jaeger, Duesseldorf (DE)

(72) Inventors: Andreas Knapp, Duesseldorf (DE); Sonja Voget, Goettingen (DE); Rolf Daniel, Goettingen (DE); Karl-Erich Jaeger, Duesseldorf (DE)

(21) Appl. No.: 14/680,255

(22) Filed: Apr. 7, 2015

(51) Int. Cl. C12N 9/20

(52) U.S. Cl. CPC C12N 9/20; C12Y 301/01003

(57) ABSTRACT

The present invention relates to the improved production of proteins, preferably enzymes such as lipases. In particular, the invention relates to a mutated signal peptide and nucleotide sequence encoding said signal peptide that results in an increased protein secretion. Further, the invention relates to a mutated promoter that results in an increased protein expression. It was surprisingly found that the combination of the mutated signal peptide and the mutated promoter act synergistically to result in an about 100-fold increased protein production. Nucleic acid molecules, expression vectors and host cells comprising the mutated signal peptide, mutated promoter, or the combination thereof are also encompassed by the invention. Finally, the invention relates to methods and uses of such nucleic acid molecules, expression vectors and host cells for protein preparation.

### FIELD OF THE INVENTION

The invention is in the field of biotechnology and aims at improving protein production. In particular, the invention relates to nucleic acid molecules and expression vectors for preparing proteins and to microorganisms comprising such nucleic acid molecules and/or expression vectors. The invention further relates to methods and uses of such nucleic acid molecules, expression vectors and host cells for protein preparation.

### BACKGROUND OF THE INVENTION

For the industrial production of proteins, for example hydrolytic enzymes, preferably host cells are used which are capable of secreting large amounts of the protein into the cell culture supernatant, since it is not necessary to disrupt the cells to release the protein. For this purpose, host cells are preferably used, for example Burkholderia species, which can be cultured using cost-effective culture media in efficient high-cell-density fermentation procedures and are capable of secreting multiple grams per liter of the target protein into the culture supernatant. The protein to be secreted may be expressed naturally in the host cell. Alternatively, the protein to be secreted may be recombinantly expressed from expression vectors which have been introduced into the host cell and which encode the protein to be secreted. The expressed protein usually comprises a signal peptide which brings about the export thereof from the host cell to the cell culture supernatant. The signal peptide is usually part of the polypeptide chain translated in the host cell, and may be posttranslationally cleaved off from the protein.

Especially for this extracellular production of heterologous proteins, there are, however, numerous bottlenecks and a corresponding high demand for optimization of the secretion processes. One of these bottlenecks is the selection of a signal peptide which allows efficient export of the target protein from the host cell. Signal peptides can, in principle, be newly combined with proteins, more particularly enzymes. For example, the publication by Brockmeier et al. ((2006) J. Mol. Biol. 362: 393-402) describes the strategy of screening a signal peptide library. However, not every signal peptide also brings about adequate export of the protein under fermentation conditions, more particularly industrial or industrial-scale fermentation conditions.

Research over the last decades focused on the development of new methods to improve enzymes by directed evolution, rational design and computational methods. Lipases as the third-largest group of commercially used enzymes represent the most important class of biocatalysts for organic synthesis. However, efficient expression and secretion of lipases is still a problem, and many biotechnologically interesting lipases, e.g. those produced by Pseudozyma aphidis (formerly Candida antarctica) or by various Pseudomonas species, can be produced in E. coli, but are not efficiently secreted from these bacteria, thus requiring optimization of the expression strains.

The bacterium Burkholderia glumae (formerly known as Pseudomonas glumae ) is a moderate plant pathogen, which causes husk rot and mildew on the shoots and panicles of rice plants. All B. glumae strains studied so far infect rice panicles and produce a phytotoxin called toxoflavin which is regulated by a LuxR-LuxI-type quorum sensing (QS) system. Like many other bacteria, B. glumae produces an extracellular lipase (triacylglycerol hydrolase, EC 3.1.1.3). This type of extracellular lipase is secreted into the culture medium, thereby facilitating down-stream processing and lowering costs. These lipases belong to the family of α/β hydrolases and catalyze the hydrolysis of triglycerides to glycerol and fatty acids. They are most frequently used as biocatalysts in organic chemistry, as they do not require cofactors, and usually show a broad substrate specificity and high enantioselectivity as well as high stability in non-aqueous media such as ionic liquids, supercritical fluids and organic solvents. Under non-aqueous reaction conditions lipases can catalyze the synthesis of various esters by esterification, interesterification, and transesterification. Additional fields of lipase application include the production of food and feed ingredients as well as intermediates for pharmaceuticals and, more recently, also for biodiesel production. B. glumae PG1 (WO 93/00924 A1) produces the extracellular lipase LipA which is used for the production of enantiopure alcohols and amines as intermediates in the synthesis of pharmaceuticals.

The production of lipases at high yield would therefore be desirable, and there is a need to improve the expression of lipases to increase the yield and expression rate. It is therefore an object of the invention to improve the production of a protein, in particular a lipase, in a host cell and, as a result, to increase the protein product yield in a fermentation procedure.

In the present invention, it was found that both a mutation in the signal peptide of LipA as well as a mutation within the lipase promoter increase the lipase production significantly. Further, it was surprisingly found that the combination of these mutations acts synergistically and results in a significantly increased lipase production and secretion.

### BRIEF SUMMARY OF THE INVENTION

The present invention relates to an isolated nucleic acid molecule comprising a nucleotide sequence that is at least 80% identical to SEQ ID NO 1 and encodes a polypeptide having a hydrophobic amino acid at a position corresponding to position 4 of the amino acid sequence as depicted in SEQ ID NO 2. In one embodiment, the hydrophobic amino acid is selected from the group consisting of leucine, valine, isoleucine, methionine and alanine, preferably it is leucine. More preferably, the isolated nucleic acid molecule has the nucleotide sequence according to SEQ ID No. 8 and encodes a protein having the amino acid sequence according to SEQ ID No. 9. In another embodiment, the invention relates to a microorganism comprising said nucleic acid molecule. In yet another embodiment, the invention relates to an expression vector comprising said nucleic acid molecule. In yet another embodiment, the invention relates to a recombinant microorganism comprising said expression vector.

The invention further relates to an isolated nucleic acid molecule comprising a nucleotide sequence that is at least 80% identical to SEQ ID NO 3 and contains at a position corresponding to position 116 of the nucleotide sequence as depicted in SEQ ID NO 3 a thymidine residue. More preferably, the isolated nucleic acid molecule has the nucleotide sequence according to SEQ ID No. 10. In one embodiment, the invention relates to a microorganism comprising said nucleic acid molecule. In another embodiment, the invention relates to an expression vector comprising said nucleic acid molecule. In yet another embodiment, the invention relates to a recombinant microorganism comprising said expression vector.

The invention further relates to an isolated nucleic acid molecule comprising a first nucleotide sequence that is at least 80% identical to SEQ ID NO 3 and a second nucleotide sequence that is located at the 3′ end of the first nucleotide sequence and is operably linked thereto and that is at least 80% identical to SEQ ID NO 1, wherein the first nucleotide sequence contains at a position corresponding to position 116 of the nucleotide sequence as depicted in SEQ ID NO 3 a thymidine residue and wherein the second nucleotide sequence encodes a polypeptide having a hydrophobic amino acid at a position corresponding to position 4 of the amino acid sequence as depicted in SEQ ID NO 2. In one embodiment, the hydrophobic amino acid is selected from the group consisting of leucine, valine, isoleucine, methionine and alanine, preferably it is leucine. More preferably, the first nucleotide sequence is depicted in SEQ ID No. 10 and the second nucleotide sequence is depicted in SEQ ID No. 8. In another embodiment, the invention relates to a microorganism comprising said nucleic acid molecule. In yet another embodiment, the invention relates to an expression vector comprising said nucleic acid molecule and to a recombinant microorganism comprising said expression vector.

Said nucleic acid molecule or the expression vector may further comprise a third nucleotide sequence coding for an enzyme, wherein the third nucleotide sequence is fused to the second nucleotide sequence, preferably wherein the enzyme is a lipase and has at least 70% identity to the amino acid sequence as depicted in SEQ ID NO 6. Said nucleic acid molecule or expression vector may further comprise a fourth nucleotide sequence coding for a chaperone, wherein the fourth nucleotide sequence is functionally linked to the third nucleotide sequence, preferably wherein the chaperone has at least 70% identity to the amino acid sequence as depicted in SEQ ID NO 7. In yet another embodiment, the invention relates to a recombinant microorganism comprising said expression vector.

In yet another embodiment, the invention relates to a method for producing a lipase, wherein the method comprises cultivating a recombinant microorganism under conditions suitable for the production of the lipase and obtaining the lipase, wherein the microorganism comprises an expression vector that comprises the third nucleotide sequence. In yet another embodiment, the invention relates to a lipase obtainable by the method.

In a particularly preferred embodiment, the isolated nucleic acid molecule comprises a nucleotide sequence as depicted in SEQ ID NO 4 comprising a first nucleotide sequence that is identical to SEQ ID NO 10 and a second nucleotide sequence which is located at the 3′ end of the first nucleotide sequence and which is identical to SEQ ID NO 8, wherein the first and the second nucleotide sequence are operably linked to each other. In one embodiment, the invention relates to a microorganism comprising said nucleic acid molecule. In another embodiment, the invention relates to an expression vector comprising said nucleic acid molecule and to a recombinant microorganism comprising said expression vector.

Said expression vector may further comprise a third nucleotide sequence coding for an enzyme, wherein the third nucleotide sequence is fused to the second nucleotide sequence, preferably wherein the enzyme is a lipase and has at least 70% identity to the amino acid sequence as depicted in SEQ ID NO 6 or is encoded by a nucleic acid sequence which is at least 70% identical to the sequence according to SEQ ID NO 12. Such an expression vector may further comprise a fourth nucleotide sequence coding for a chaperone, wherein the fourth nucleotide sequence is functionally linked to the third nucleotide sequence, preferably wherein the chaperone has at least 70% identity to the amino acid sequence as depicted in SEQ ID NO 7 or is encoded by a nucleic acid sequence which is at least 70% identical to the sequence according to SEQ ID NO 13. In yet another embodiment, the invention relates to a recombinant microorganism comprising said expression vector. In yet another embodiment, the invention relates to a method for producing a lipase, wherein the method comprises cultivating said recombinant microorganism under conditions suitable for the production of the lipase and obtaining the lipase, wherein the microorganism comprises an expression vector that comprises the third nucleotide sequence. In yet another embodiment, the invention relates to a lipase obtainable by said method.

In another particularly preferred embodiment, the isolated nucleic acid molecule comprises a nucleotide sequence as depicted in SEQ ID NO 5 or SEQ ID NO 11, comprising a first nucleotide sequence that is identical to SEQ ID NO 10, a second nucleotide sequence that is identical to SEQ ID NO 8 and is located at the 3′ end of the first nucleotide sequence and in operable linkage thereto, a third nucleotide sequence that is identical to SEQ ID NO 12, and a fourth nucleotide sequence that is identical to SEQ ID NO 13. In one embodiment, the invention relates to a microorganism comprising said nucleic acid molecule. In another embodiment, the invention relates to a method for producing a lipase, wherein the method comprises cultivating said microorganism under conditions suitable for the production of the lipase and obtaining the lipase. In yet another embodiment, the invention relates to a lipase obtainable by said method. In yet another embodiment, the invention relates to the use of said nucleic acid molecule for the production of a lipase. In yet another embodiment, the invention relates to an expression vector comprising said nucleic acid molecule. In yet another embodiment, the invention relates to a recombinant microorganism comprising said expression vector. In yet another embodiment, the invention relates to a method for producing a lipase, wherein the method comprises cultivating said recombinant microorganism under conditions suitable for the production of the lipase and obtaining the lipase. In yet another embodiment, the invention relates to a lipase obtainable by said method.

In one aspect of the invention, the microorganism or the recombinant microorganism is a bacterium selected from the group consisting of Burkholderia glumae, Burkholderia gladioli, Burkholderia mallei, Burkholderia pseudomallei, Burkholderia thailandensis, Escherichia coli, Bacillus licheniformis, Bacillus subtilis, Bacillus lentus, Bacillus amyloliquefaciens, Bacillus alcalophilus, Bacillus globigii, Bacillus gibsonii, Bacillus clausii, Bacillus halodurans and Bacillus pumilus.

The invention further relates to a recombinant protein comprising a polypeptide sequence, wherein the polypeptide sequence is at least 90% identical to SEQ ID NO 2 and has a hydrophobic amino acid at a position corresponding to position 4 of the amino acid sequence as depicted in SEQ ID NO 2. Preferably, the hydrophobic amino acid is selected from the group consisting of leucine, valine, isoleucine, methionine and alanine. Most preferably the hydrophobic amino acid is leucine.

### BRIEF DESCRIPTION OF THE DRAWINGS

FIG. 1 Lipase production of B. glumae PG1 wild-type (PG1) and B. glumae LU8093 A: Relative lipase activity in the supernatant (SN) and cell extract (CE). LipA was detected in culture supernatants (SN LipA) and LipB was detected in cell extract (CE LipB) by Western blotting after SDS-PAGE. Samples of 10 μl were loaded into each lane corresponding to a cell density of OD 580 nm=5 for cell extracts and=50 for supernatants. B: Relative change of lipA and lipB transcript levels in B. glumae LU8093 compared to the wild-type B. glumae PG1 (arbitrarily set as 1).

FIG. 2 Two mutations identified by comparative genome sequencing and localized to the lipAB operon of B. glumae LU8093. The first mutation is located in the lipAB promoter region (PlipAB) and is present in the constructed variant lipAB-1; the second mutation located in the LipA signal peptide coding sequence is present in the constructed variant lipAB-2; variant lipAB-3 contains both mutations. Two putative binding sites for δ54 transcription factors and the transcription start (30 1) are underlined in the DNA sequence shown below. Coding triplets no. 1-7 of the lipA signal peptide are translated into the corresponding amino acid sequence, and mutations identified in B. glumae LU8093 are marked with asterisks. The amino acid exchange resulting from mutation lipAB-2 is indicated in the amino acid sequence.

FIG. 3 Expression of different lipase operons in B. glumae PG1ΔlipAB.: Relative lipase activity in cell-free supernatants (SN) and cell extracts (CE). LipA in supernatants (SN LipA) and LipB in cell extracts (CE LipB) were detected by Western blotting after SDS-PAGE with each lane containing 10 μl sample corresponding to a cell density of O.D. 580 nm=5 for cell extracts and=50 for supernatants.

### DETAILED DESCRIPTION OF THE INVENTION

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)”, “i”, “ii” etc. and the like in the description and in the claims, are used for distinguishing between similar elements and not necessarily for describing a sequential or chronological order. In case the terms relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The present invention relates to the improved production of proteins. In particular, the invention relates to a mutated signal peptide and nucleotide sequence encoding said signal peptide that results in an increased protein secretion from a host cell. Further, the invention relates to a mutated promoter that results in an increased protein expression in a host cell. It was surprisingly found that the combination of said mutated signal peptide and said mutated promoter acts synergistically to result in an about 100-fold increased protein production. Expression vectors and host cells comprising the mutated signal peptide, mutated promoter, or the combination thereof are also encompassed by the invention. Finally, the invention relates to methods and uses of such nucleic acid molecules, expression vectors and microorganisms for protein preparation.

Expression is the process by which information from a gene is used in the synthesis of a functional gene product, such as a protein. For the purposes of the present invention, expression means the biosynthesis of ribonucleic acid (RNA) and proteins from the genetic information provided by a nucleic acid molecule of the present invention. Generally, gene expression comprises the transcription, i.e., the synthesis of a messenger ribonucleic acid (mRNA) on the basis of the DNA (deoxyribonucleic acid) sequence of a gene or a nucleotide sequence of the invention, and the translation of the mRNA into the corresponding polypeptide chain, which in some organisms may additionally be modified posttranslationally. The expression of a protein consequently describes the biosynthesis thereof from the genetic information which according to the invention is provided in a nucleic acid molecule or on an expression vector.

Within the meaning of the present invention, “sequence identity” denotes the degree of conformity with regard to the 5′-3′ sequence within a nucleic acid molecule in comparison to another nucleic acid molecule or the degree of conformity with regard to the N-terminal to C-terminal sequence within an amino acid molecule in comparison to another amino acid molecule. The sequence identity may be determined using a series of programs, which are based on various algorithms, such as BLASTN, ScanProsite, the laser gene software, etc. As an alternative, the BLAST program package of the National Center for Biotechnology Information (http://www.ncbi.nlm.nih.gov/) may be used with the default parameters. In addition, the program Sequencher (Gene Codes Corp., Ann Arbor, Mich., USA) using the “dirtydata”-algorithm for sequence comparisons may be employed.

Such a sequence comparison makes it possible to reveal the similarity of the compared sequences to one another. It is usually reported in percent identity, i.e., the proportion of identical nucleotides or amino acid residues on the same positions or positions corresponding to one another in an alignment.

Preferably, the identity values provided in the present application refer to the entire length of the various indicated nucleotide or amino acid sequences.

By aligning two nucleotide or amino acid sequences it is also possible to identify corresponding nucleotides or amino acids, i.e. nucleotides or amino acids which are in the same sequence context as a specific nucleotide or amino acid in the reference sequence, but do not necessarily have the same numbering as said nucleotide or amino acid in the reference sequence.

A “nucleic acid molecule” is composed of nucleotides and may be used to code for polypeptides or proteins or biologically active fragments thereof.

An “isolated” nucleic acid molecule is separated from other nucleic acid molecules that are present in the natural source of the nucleic acid and can moreover be substantially free from other cellular material or culture medium, if it is being produced by recombinant techniques, or can be free from chemical precursors or other chemicals, if it is being synthesized chemically.

A nucleic acid molecule can be isolated by means of standard techniques of molecular biology and the sequence information provided. For example, cDNA can be isolated from a suitable cDNA library, using one of the concretely disclosed complete sequences or a segment thereof as hybridization probe and standard hybridization techniques (as described for example in Sambrook et al., Molecular Cloning: A Laboratory Manual. 2nd edition, Cold Spring Harbor Laboratory, Cold Spring Harbor Laboratory Press, Cold Spring Harbor, N.Y., 1989). In addition, a nucleic acid molecule comprising one of the disclosed sequences or segments thereof can be isolated by the polymerase chain reaction, using oligonucleotide primers that were constructed on the basis of this sequence. The nucleic acid molecule amplified in this way may be cloned in a suitable vector and characterized by DNA sequencing. Oligonucleotides may also be produced by standard methods of synthesis, e.g. using an automatic DNA synthesizer. Nucleic acid molecules according to the invention can for example be isolated by usual hybridization techniques or the PCR technique from bacteria, e.g. via genomic or cDNA libraries.

The terms “polypeptide” and “protein” are used interchangeably herein and refer to a biomolecule which is composed of amino acids. The specific order of the amino acids within the polypeptide or protein is determined by the encoding nucleic acid sequence and is called amino acid sequence. The term “polypeptide” is not limited by a minimum number of amino acids present in it.

The term “hydrophobic amino acid”, as used herein, is intended to mean amino acids that have hydrophobic side chains. Amino acids having hydrophobic side chains include, but are not limited to, leucine (Leu), glycine (Gly), alanine (Ala), valine (Val), isoleucine (Ile), proline (Pro), phenylalanine (Phe), methionine (Met), and tryptophan (Trp). It is particularly preferred that the hydrophobic amino acid at a position corresponding to position 4 of the amino acid sequence as depicted in SEQ ID NO 2 is selected from the group consisting of leucine, valine, isoleucine, methionine and alanine. It is particularly preferred that the hydrophobic amino acid is leucine.

A “signal peptide”, as used herein, refers to a short peptide (usually about 5-30 amino acids) present at the terminus of newly synthesized proteins. The signal peptide promotes the secretion of the protein to which it is fused via a secretory pathway. Preferably, the signal peptide promotes the secretion of the protein into the cell culture supernatant of a cell culture comprising a microorganism.

The term “signal peptide according to the invention” refers to a peptide having an amino acid sequence which is at least 90% identical to SEQ ID No. 2 and has a hydrophobic amino acid at a position corresponding to position 4 of the amino acid sequence as depicted in SEQ ID No. 2. Preferably, the hydrophobic amino acid is selected from the group consisting of leucine, valine, isoleucine, methionine and alanine, more preferably it is leucine. With increasing preference, the amino acid sequence of the signal peptide is at least 91%, 92%, 93%, 94%, 95%, 96%, 97%, 98%, 99% and very particularly preferably 100% identical to the amino acid sequence as depicted in SEQ ID NO 2. When calculating the percent sequence identity to SEQ ID NO 2 the hydrophobic amino acid at position 4 is not taken into account, i.e. an amino acidsequence corresponding to SEQ ID NO 2 except for position 4 (e.g. a leucine instead of serine) would according to the meaning of the invention be an amino acid sequence sequence that is 100% identical to SEQ ID NO 2. Most preferably, the signal peptide sequence according to the invention is the amino acid sequence according to SEQ ID No. 9.

The signal peptide according to the present invention is encoded by a nucleotide sequence that is at least 80% identical to SEQ ID NO 1 and encodes a polypeptide having a hydrophobic amino acid at a position corresponding to position 4 of the amino acid sequence as depicted in SEQ ID NO 2. It is particularly preferred that the hydrophobic amino acid is selected from the group consisting of leucine, valine, isoleucine, methionine and alanine, preferably it is leucine. With increasing preference, the nucleotide sequence encoding the signal peptide sequence according to the invention is at least 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the nucleotide sequence as depicted in SEQ ID NO 1. When calculating the percent sequence identity to SEQ ID NO 1 the mutation resulting in a hydrophobic amino acid at position 4 is not taken into account, i.e. a nucleotide sequence corresponding to SEQ ID NO 1 except for the nucleotides coding for the amino acid at position 4 (e.g. a leucine instead of serine) of the corresponding protein would according to the meaning of the invention be a nucleotide sequence that is 100% identical to SEQ ID NO 1. Most preferably, the nucleic acid sequence encoding the signal peptide of the present invention is the nucleic acid sequence according to SEQ ID NO. 8.

It is to be understood that deviations from the nucleotide sequence as depicted in SEQ ID NO 1 resulting in a nucleotide sequence that is at least 80% identical to the nucleotide sequence as depicted in SEQ ID NO 1 will not result in a loss of the function of the encoded signal peptide, i.e. the amino acid sequence encoded by such an nucleotide sequence will still be capable of effecting the secretion of a protein fused to this amino acid sequence. The amount of protein secreted by a signal peptide encoded by a nucleotide sequence which is at least 80% identical to SEQ ID No.1 or by a signal peptide having an amino acid sequence which is at least 80% identical to SEQ ID NO 2 is at least 30%, 35%, 40%, 45% or 50%, preferably at least 55%, 60%, 65%, 70%, 75% or 80%, more preferably at least 82%, 84%, 86%, 88% or 90% and most preferably at least 91%, 92%, 93%, 94%, 95%, 96%, 97%, 98% or 99% of the amount of the same protein secreted by a signal peptide according to SEQ ID No. 9 which is encoded by SEQ ID No. 8.

In one embodiment, the signal peptide according to the invention is fused to a protein to be secreted.

The term “fused” is intended to mean that the signal peptide according to the invention is linked to the amino acid sequence of the protein to be secreted by a peptide bond. Such a fusion protein may have the following structure: N-terminus-signal peptide-protein amino acid sequence-C-terminus. Such a structure of the protein to be expressed has been found to be particularly advantageous. It is however also encompassed that a connecting sequence (also “coupler” or “spacer”) is arranged between the signal peptide and the amino acid sequence of the protein. Hence, the fusion protein may also have the structure: N-terminus-signal peptide-connecting sequence-protein amino acid sequence-C-terminus. Such a structure of the protein to be expressed has likewise been found to be particularly advantageous. Preferably, the length of the connecting sequence is between 1 and 50 amino acids, between 2 and 25 amino acids, between 2 and 15 amino acids, between 3 and 10 amino acids, and particularly preferably between 3 and 5 amino acids.

The term “protein to be secreted” refers to an enzyme, preferably an esterase, more preferable a hydrolase, even more preferably a hydrolase selected from the group consisting of lipase, phospholipase, cholinesterase, acetylcholinesterase, butyrylcholinesterase, pectinesterase 6-phosphogluconolactonase, or PAF acetylhydrolase, and most preferably a lipase. In a preferred embodiment, the lipase is an extracellular lipase. The term “extracellular lipase”, as used herein, denotes in particular those lipases in enzyme class E.C. 3.1.1.3. In another preferred embodiment, the extracellular lipase is produced by bacteria of the genus Burkholderia, preferably by Burkholderia glumae. In a particularly preferred embodiment the extracellular lipase is LipA of Burkholderia glumae. It is therefore particularly preferred that the protein is a lipase that has at least 70% identity to the amino acid sequence as depicted in SEQ ID NO 6. With increasing preference, the lipase comprises an amino acid sequence which is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the amino acid sequence as depicted in SEQ ID NO 6. A variant of the lipase which has a sequence identity of at least 70% to the amino acid sequence as depicted in SEQ ID No. 6 or which is encoded by a nucleic acid sequence having a sequence identity of at least 70% to the nucleic acid sequence according to SEQ ID NO 12 has essentially the same activity as the lipase according to SEQ ID No. 6. With respect to the lipase the term “essentially the same activity” means that the lipase variant has an activity which is at least 30%, 35%, 40%, 45% or 50%, preferably at least 55%, 60%, 65%, 70% or 75%, more preferably at least 80%, 82%, 84%, 86% or 88% and most preferably at least 90%, 91%, 92%, 93%, 94%, 95%, 96%, 97%, 98% or 99% of the lipase activity of the lipase according to SEQ ID No. 6. The skilled person knows how to determine the lipase activity and a suitable method is described in the examples section herein.

In another embodiment, the isolated nucleic acid molecule encoding a signal peptide sequence according to the invention, may further comprise a promoter operably linked to the signal peptide sequence, in particular a promoter sequence according to the invention. Such a nucleic acid molecule may additionally also comprise a nucleotide sequence coding for a protein to be secreted as described above.

The term “operably linked” refers to the association of nucleic acid sequences on a single nucleic acid fragment so that the function of one is affected by the other. In the context of a promoter the term means that the coding sequence is under the transcriptional control of the promoter such that the promoter regulates the transcription and consequently the expression of the coding sequence. In the present invention the nucleotide sequence encoding the signal peptide and/or the protein to be secreted is operably linked to the promoter sequence of the present invention.

A “promoter” is understood to mean a DNA sequence which allows the regulated expression of a gene. A promoter sequence is naturally a component of a gene and is often located at the 5′ end thereof and thus upstream of the RNA-coding region. Preferably, in a nucleic acid molecule according to the invention the promoter sequence is located 5′ upstream of the nucleotide sequence encoding the signal peptide and/or the protein to be secreted. The most important property of a promoter is the specific interaction with at least one DNA-binding protein or polypeptide which mediates the start of the transcription of the gene and which is referred to as a transcription factor. Multiple transcription factors and/or further proteins are frequently involved at the start of the transcription. A promoter is therefore preferably a DNA sequence having promoter activity, i.e., a DNA sequence to which at least one transcription factor binds at least transiently in order to initiate the transcription of a gene by an RNA polymerase. The strength of a promoter is measurable via the transcription rate of the expressed gene, i.e., via the number of RNA molecules, more particularly mRNA molecules, generated per unit time.

The term “promoter sequence according to the invention” refers to a nucleotide sequence that is at least 80% identical to SEQ ID NO 3 and contains at a position corresponding to position 116 of the nucleotide sequence depicted in SEQ ID NO 3 a thymidine residue. With increasing preference, the promoter sequence according to the invention is at least 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the nucleotide sequence as depicted in SEQ ID NO 3. When calculating the percent sequence identity to SEQ ID NO 3 the position 116 is not taken into account, i.e. a nucleotide sequence corresponding to SEQ ID NO 3 except for position 116 (e.g. thymidine a instead of cytidine) would according to the meaning of the invention be a nucleotide sequence that is 100% identical to SEQ ID NO 3. In the most preferred embodiment the promoter sequence according to the present invention has the nucleotide sequence according to SEQ ID No. 10.

It is to be understood that deviations from the nucleotide sequence as depicted in SEQ ID NO 3 resulting in a nucleotide sequence that is at least 80% identical to the nucleotide sequence as depicted in SEQ ID NO 3 will not result in a loss of the function as promoter, i.e. the nucleotide sequence will still be capable of regulating the expression of the nucleotide sequence encoding the signal peptide or protein to be secreted, i.e. it has essentially the same activity as the promoter sequence according to SEQ ID No. 3.

The skilled person knows how to determine the promoter activity and to compare the activities of different promoters. For this purpose, the promoters are typically operably linked to a nucleic acid sequence encoding a reporter protein such as luciferase, green fluorescence protein or beta-glucuronidase and the activity of the reporter protein is determined, optionally in comparison to the activity of one more other promoters. Alternatively or additionally, the mRNA levels of the endogenous genes operably linked to the promoter of the wildtype organism can be compared with each other, e.g. by quantitative real time PCR or Northern Blot.

The term “essentially the same activity” refers to promoter sequences which have at least 50% or 55%, preferably at least 60, 65 or 70%, more preferably at least 75, 80, 85 or 90% and most preferably at least 92, 94, 96, 98 or 99% of the promoter activity of the promoter according to SEQ ID NO. 3, i.e. the activity of the reporter protein under the control of the promoter having essentially the same activity as the promoter of SEQ ID No. 3 is at least 50% or 55%, preferably at least 60, 65 or 70%, more preferably at least 75, 80, 85 or 90% and most preferably at least 92, 94, 96, 98 or 99% of the activity of the reporter protein under the control of the promoter according to SEQ ID No. 3.

The isolated nucleic acid molecule comprising a promoter sequence according to the invention may further comprise a nucleotide sequence coding for a protein to be secreted as described above operably linked to the promoter sequence according to the invention. Such a nucleic acid molecule may additionally also comprise a signal peptide sequence according to the invention. It is particularly preferred that in such a nucleic acid molecule the signal peptide sequence is fused to the nucleotide sequence coding for a protein to be secreted.

In another preferred aspect, the invention relates to an isolated nucleic acid molecule comprising a first nucleotide sequence and a second nucleotide sequence located at the 3′ end of the first nucleotide sequence and operably linked thereto, wherein the first nucleotide sequence is a a promoter sequence according to the invention and the second nucleotide sequence is a signal peptide sequence according to the invention.

The “first nucleotide sequence”, as used herein, is intended to mean the promoter sequence according to the invention. The “second nucleotide sequence”, as used herein, is intended to mean the signal peptide sequence according to the invention. The “third nucleotide sequence”, as used herein, is intended to mean a nucleotide sequence coding for a protein to be secreted, preferably an enzyme, more preferably a lipase as defined herein. The “fourth nucleotide sequence”, as used herein, is intended to mean a nucleotide sequence coding for a chaperone as defined herein.

The term “located at the 3′ end”, as used herein, is intended to mean that the second nucleotide sequence is situated 3′ downstream of the first nucleotide sequence in the nucleic acid molecule (in the 5′→3′ orientation) and is operably linked thereto. A further nucleotide sequence, such as a nucleotide linker, may be located between the first nucleotide sequence and the second nucleotide sequence. It is preferred that there are no nucleotide sequences between the first and second sequences which reduce the expression rate of the second nucleotide sequence that is fused to a nucleotide sequence coding for a protein to be secreted.

Thus, in one embodiment, the first nucleotide sequence is located at the 5′ end of the second nucleotide sequence, wherein a nucleotide linker is present between the 3′ end of the first nucleotide sequence and the 5′ end of the second nucleotide sequence. The nucleotide linker may comprise a 5′ end untranslated region.

It was surprisingly found that the combination of the first and second nucleotide sequence acts synergistically and results in a significant increase in the production of a protein, in particular a lipase and more particular the LipA lipase. In particular, the expression and secretion of the protein, in particular a lipase and more particular the LipA lipase, were increased in an unforeseeable extent.

The increase of protein production may be determined by determining the protein amount in the supernatant and/or the cell extract of a microorganism according to the invention comprising the promoter sequence of the present invention and the signal peptide of the present invention and comparing said protein amount to the protein amount in the supernatant and/or cell extract of a microorganism not comprising the promoter sequence of the present invention and the signal peptide of the present invention. In one embodiment, the protein amount is increased by about 10-fold, 20-fold, 30-fold, 40-fold, 50-fold, 60-fold, 70-fold, 80-fold, 90-fold, 100-fold, 110-fold, 120-fold, 130-fold, or 140-fold or more compared to a microorganism not comprising the promoter sequence of the present invention and the signal peptide of the present invention. In one embodiment, the protein amount in a microorganism comprising the signal peptide of the present invention, but not the promoter region of the present invention sequence is increased by about 4-fold, 5-fold, 6-fold, 7-fold, 8-fold, 9-fold, or 10-fold or more compared to a microorganism not comprising the signal peptide of the present invention. In another embodiment, the protein amount in a microorganism comprising the promoter sequence of the present invention, but not the signal peptide of the present invention sequence is increased by about 10-fold, 20-fold, 30-fold, 35-fold, 40-fold, 45-fold, or 50-fold compared to a microorganism not comprising the promoter sequence of the present invention.

If the protein expressed with the signal peptide of the present invention and the promoter sequence of the present invention is a lipase, the increase of protein production resulting from the combination of the signal peptide sequence of the present invention and the promoter sequence of the present invention results in an about 90-fold, 100-fold, 110-fold, 120-fold, 130-fold, 140-fold, or 150-fold increased lipase activity.

Thus, in a particularly preferred embodiment the invention provides an isolated nucleic acid molecule comprising a first nucleotide sequence and a second nucleotide sequence located at the 3′ end of the first nucleotide sequence, wherein the first nucleotide sequence is shown in SEQ ID No. 10 and the second nucleotide sequence is depicted in SEQ ID NO 8.

In another preferred embodiment, the isolated nucleic acid molecule comprises a nucleotide sequence that is at least 80% identical to SEQ ID NO 4, wherein the nucleotide sequence as depicted in SEQ ID NO 4 comprises the first nucleotide sequence and the second nucleotide sequence located at the 3′ end of the first nucleotide sequence. With increasing preference, the isolated nucleic acid molecule comprises a nucleotide sequence which is at least 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the nucleotide sequence specified in SEQ ID NO 4. Thus, in a particularly preferred embodiment the isolated nucleic acid molecule comprises a nucleotide as depicted in SEQ ID NO 4.

The isolated nucleic acid molecule comprising a nucleotide sequence as depicted in SEQ ID NO 4 may further comprise a third nucleotide sequence coding for a protein to be secreted as described above, preferably a lipase, more preferably a lipase according to SEQ ID No. 6 or a variant thereof having at least 70% sequence identity to the sequence according to SEQ ID No. 6, operably linked to the nucleotide sequence as depicted in SEQ ID NO 4, and/or a fourth nucleotide sequence coding for a chaperon.

Thus, in yet another preferred embodiment, the isolated nucleic acid molecule comprises a nucleotide sequence that is at least 70% identical to SEQ ID NO 5 or SEQ ID No. 11. With increasing preference, the isolated nucleic acid molecule comprises a nucleotide sequence which is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the nucleotide sequence specified in SEQ ID NO 5 or SEQ ID No. 11. Thus, in a particular preferred embodiment, the isolated nucleic acid molecule comprises a nucleotide sequence as depicted in SEQ ID NO 5 or SEQ ID No. 11.

In a further aspect, the invention relates to a recombinant protein comprising a polypeptide sequence, wherein the polypeptide sequence is at least 90% identical to SEQ ID NO 2 and has a hydrophobic amino acid at a position corresponding to position 4 of the amino acid sequence as depicted in SEQ ID NO 2. The hydrophobic amino acid is preferably selected from the group consisting of leucine, valine, isoleucine, methionine and alanine, particularly preferably the hydrophobic amino acid is leucine.

In yet a further aspect, the invention relates to an expression vector comprising a nucleic acid molecule of the invention.

Expression vectors are extrachromosomal genetic elements consisting of nucleic acids, preferably deoxyribonucleic acid (DNA), and are known to a person skilled in the art in the field of biotechnology. Particularly when used in bacteria, they are specific plasmids, i.e., circular genetic elements. The expression vectors can, for example, include those which are derived from bacterial plasmids, from viruses or from bacteriophages, or predominantly synthetic expression vectors or plasmids containing elements of very diverse origin. With the further genetic elements present in each case, expression vectors are capable of establishing themselves in host cells, into which they have been introduced preferably by transformation, over multiple generations as stable units. In this respect, it is insignificant for the purposes of the invention whether they are established extrachromosomally as separate units or are integrated into a chromosome or chromosomal DNA. Which of the numerous systems is chosen depends on the individual case. Critical factors may, for example, be the achievable copy number, the selection systems available, including especially the antibiotic resistances, or the culturability of the host cells capable of vector uptake.

An expression vector further comprises at least one nucleotide sequence, preferably DNA, having a control function for the expression of the nucleotide sequence coding for the signal peptide and/or protein (a so-called gene regulatory sequence). A gene regulatory sequence is, in this case, any nucleotide sequence which, through its presence in the particular host cell, affects, preferably increases, the transcription rate of the nucleotide sequence coding for the signal peptide and/or protein. Preferably, it is a promoter sequence, since such a sequence is essential for the expression of the nucleotide sequence of the signal peptide and/or protein. However, an expression vector according to the invention can also comprise yet further gene regulatory sequences, for example one or more enhancer sequences. An expression vector for the purposes of the invention consequently comprises at least one functional unit composed of the nucleotide sequence coding for a signal peptide and/or protein and a promoter (expression cassette). It can, but need not necessarily, be present as a physical entity. The presence of at least one promoter is consequently essential for an expression vector according to the invention. It is preferred that the promoter is the promoter sequence according to the invention.

Preferably, the promoter sequence according to the invention and the signal peptide sequence according to the invention and/or a nucleotide sequence coding for a protein to be secreted are operably linked to each other on the expression vector, i.e. the promoter sequence is located at the 5′ end of the nucleotide sequence coding for a signal peptide and/or protein to be secreted as described above.

In one embodiment, the expression vector further comprises a third nucleotide sequence coding for an enzyme, wherein the third nucleotide sequence is fused to the second nucleotide sequence. Preferably, the enzyme is a lipase, more preferably a lipase according to SEQ ID No. 6 or a variant thereof having at least 70% sequence identity to the sequence according to SEQ ID No. 6. Also preferably, the lipase is encoded by a nucleic acid sequence according to SEQ ID NO 12 or a nucleic acid sequence which is 70% identical to the nucleic acid sequence according to SEQ ID NO 12.

The expression vector may additionally comprise a fourth nucleotide sequence coding for a chaperone, wherein the fourth nucleotide sequence is functionally linked to the third nucleotide sequence. With respect to the chaperone (encoded by the fourth nucleotide sequence) and the protein to be secreted (encoded by the third nucleotide sequence), the term “functionally linked” is intended to mean that the nucleotide sequences are arranged in a manner that allows for the correct folding of the protein encoded by the third nucleotide sequence.

The “fourth nucleotide sequence”, as used herein, refers to a nucleotide sequence coding for a chaperone. A “chaperone” refers to a protein that assists the covalent folding and the assembly of the protein to be secreted. A “chaperone according to the invention” refers to a foldase which has at least 70% identity to the amino acid sequence as depicted in SEQ ID NO 7. With increasing preference, the foldase comprises an amino acid sequence which is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the amino acid sequence as depicted in SEQ ID NO 7. Thus, it is particularly preferred that the foldase is LipB of Burkholderia glumae (SEQ ID NO 7). The chaperone is encoded by a nucleic acid sequence which has at least 70% identity to the nucleic acid sequence according to SEQ ID NO 13. With increasing preference, the nucleic acid sequence encoding the chaperone is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and very particularly preferably 100% identical to the amino acid sequence as depicted in SEQ ID NO 13.

In a particularly preferred embodiment, the protein to be secreted is the lipase LipA of Burkholderia glumae according to SEQ ID NO 6 and the chaperone is the foldase LipB of Burkholderia glumae according to SEQ ID NO 7. In this embodiment, the nucleis acid sequence encoding LipB is located at the 3′ end of the nucleic acid sequence encoding LipA.

Nucleic acid molecules and expression vectors according to the invention can be prepared by commonly known methods. Such methods are, for example, presented in relevant manuals such as the one by Fritsch, Sambrook and Maniatis, “Molecular cloning: a laboratory manual”, Cold Spring Harbor Laboratory Press, New York, 1989, and familiar to a person skilled in the art in the field of biotechnology. Examples of such methods are chemical synthesis or the polymerase chain reaction (PCR), optionally in conjunction with further standard methods in molecular biology and/or chemistry or biochemistry.

In yet a further aspect, the invention relates to microorganisms comprising a nucleic acid molecule of the invention or an expression vector of the invention.

An expression vector according to the invention is preferably introduced into the host cell by the transformation thereof. The term “transformation” refers to the transfer of a genetic element, typically of a nucleic acid molecule, e.g. extrachromosomal elements such as vectors or plasmids into microorganisms. Conditions for the transformation of microorganisms and corresponding techniques are known to the person skilled in the art. These techniques include chemical transformation, ballistic impact transformation, electroporation, microinjection, or any other method that introduces the gene or nucleic acid molecule of interest into the microorganism.

This is preferably carried out by transforming an expression vector according to the invention into a microorganism, which then constitutes a recombinant microorganism according to the invention.

The term “microorganism” is intended to mean a prokaryotic or eukaryotic microorganism which preferably can be genetically manipulated, for example with regard to transformation with the expression vector and the stable establishment thereof. Preferred microorganisms are easily manipulatable from a microbiological and biotechnological perspective. This concerns, for example, ease of culture, high growth rates, low demands on fermentation media, and good production and secretion rates for foreign proteins. Microorganisms may be regulatable in terms of their activity owing to genetic regulatory elements which, for example, are made available on the vector, but may also be present in said cells before introducing the vector. For example, they can be stimulated to express a protein by controlled addition of chemical compounds serving as activators, by changing the culture conditions, or upon attainment of a particular cell density. This allows economical production of the proteins. Microorganisms can furthermore be modified with respect to their requirements in terms of culture conditions, can have selection markers, or can express additional proteins. Preferably, microorganisms secrete the expressed proteins into the medium surrounding them.

Preferably the microorganism is a prokaryotic microorganism such as bacteria. Bacteria have short generation times and low demands in terms of culture conditions. As a result, it is possible to establish cost-effective methods for protein production. In addition, a wealth of experience is available to a person skilled in the art in the case of bacteria in fermentation technology. For a specific production process, gram-negative or gram-positive bacteria may be suitable for a very wide variety of different reasons which are to be determined experimentally on an individual basis, such as nutrient sources, rate of product formation, time requirement, etc. In the case of gram-negative bacteria, for example Escherichia coli, a multiplicity of polypeptides are secreted into the periplasmic space, i.e., into the compartment between the two membranes encasing the cells. This may be advantageous for specific applications. Furthermore, it is also possible to configure gram-negative bacteria in such a way that they secrete the expressed polypeptides not only into the periplasmic space, but also into the medium surrounding the bacterium. By contrast, gram-positive bacteria, for example Burkholderia or Bacilli, do not have an outer membrane, and so secreted proteins are immediately released into the medium surrounding the bacteria, generally the culture medium, from which the expressed polypeptides can be purified. They can be isolated directly from the medium or processed further.

In a preferred embodiment, the microorganism is selected from the group of genera of Burkholderia, Escherichia, Bacillus, Klebsiella, Staphylococcus, Pseudomonas, Corynebacterium, Arthrobacter and Streptomyces, preferably is Burkholderia, Escherichia or Bacillus, most preferably Burkholderia. In a further preferred embodiment the microorganism is a bacterium selected from the group consisting of Burkholderia glumae, Burkholderia gladioli, Burkholderia mallei, Burkholderia pseudomallei, Burkholderia thailandensis, Escherichia coli, Bacillus licheniformis, Bacillus subtilis, Bacillus lentus, Bacillus amyloliquefaciens, Bacillus alcalophilus, Bacillus globigii, Bacillus gibsonii, Bacillus clausii, Bacillus halodurans and Bacillus pumilus. Most preferably the microorganism is Burkholderia glumae.

The microorganism may also be a eukaryotic microorganism such as a yeast or a unicellular fungus. Examples of preferred unicellular fungi include, but are not limited to, Aspergillus, Trichoderma, Ashbya, Neurospora, Fusarium, Beauveria. Examples of preferred yeasts include, but are not limited to, Candida, Saccharomyces, Hansenula or Pichia, especially preferred are Saccharomyces cerevisiae or Pichia pastoris. Eukaryotic microorganisms are capable of posttranslationally modifying the protein formed. This may be particularly advantageous if, for example, the proteins are to undergo, in conjunction with their synthesis, specific modifications, which is allowed by such systems.

Microorganisms according to the invention may comprise a nucleic acid molecule of the invention, for example by introduction of an expression vector of the invention into said microorganism, thereby creating a “recombinant microorganism”. In one embodiment, an expression vector of the invention is introduced into the microorganism, preferably into a microorganism of the genus Burkholderia, Escherichia, Bacillus, Pichia or Saccharomyces.

Microorganisms according to the invention are cultured and fermented in a manner known per se, for example in batch systems or continuous systems. In the first case, an appropriate culture medium is inoculated with the microorganism and the product is harvested from the medium after a period to be determined experimentally. Continuous fermentation procedures involve attaining a steady state in which, over a comparatively long period, cells partly die but also grow again and product can be removed at the same time from the medium.

In a further aspect of the invention, the microorganisms according to the invention are used to produce a protein. Preferably, the protein produced by the method of the invention is encoded by the nucleotide sequence coding for a protein to be secreted as defined above. More preferably, the protein produced is a lipase and most preferably it is the lipase according to SEQ ID No. 6 or a variant thereof having an amino acid sequence with at least 70% sequence identity to the amino acid sequence according to SEQ ID No. 6.

The invention therefore provides a method for producing a protein, comprising cultivating a microorganism according to the invention under conditions suitable for the production of the protein. In one embodiment, the method further comprises isolating the protein from the culture medium or from the microorganism. The method may further comprise the purification of the protein.

The method for producing a protein preferably comprises fermentation methods. Fermentation methods are known per se from the prior art and constitute the actual industrial-scale production step, generally followed by an appropriate purification method for the protein. The various optimal conditions for the method of production, more particularly the optimal culture conditions for the microorganism used, must be determined experimentally according to the knowledge of a person skilled in the art, for example with respect to fermentation volume and/or media composition and/or oxygen supply and/or stirrer speed.

In a preferred embodiment, the invention relates to a method for producing a lipase, wherein the method comprises cultivating a microorganism of the invention under conditions suitable for the production of the lipase and obtaining the lipase, wherein the microorganism comprises a nucleotide sequence coding for a lipase. More preferably the lipase is the lipase according to SEQ ID No. 6 or a variant thereof having an amino acid sequence with at least 70% sequence identity to the amino acid sequence according to SEQ ID No. 6.

In yet another aspect, the invention relates to a lipase obtainable by the method of the invention.

The lipase obtainably by the method of the invention may be used in numerous applications including the production of food and feed ingredients, as well as intermediates for pharmaceuticals, and for biodiesel production.

In a final aspect, the invention relates to the use of a nucleic acid molecule according to the invention, an expression vector according to the invention or a microorganism according to the invention for the production of a protein, preferably a protein encoded by the nucleotide sequence coding for a protein to be secreted as defined above, more preferably a lipase and most preferably a lipase according to SEQ ID No. 6 or a variant thereof having an amino acid sequence with at least 70% sequence identity to the amino acid sequence according to SEQ ID No. 6.

The following examples and figures are provided for illustrative purposes. It is thus understood that the examples and figures are not to be construed as limiting. The skilled person in the art will clearly be able to envisage further modifications of the principles laid out herein.

### EXAMPLES

Material and methods

Bacterial strains and growth conditions. E. coli strains DH5α and S17-1 were cultivated in LB medium (Carl Roth, Karlsruhe, Germany) at 37° C. B. glumae LU8093, B. glumae PG1 wild-type (Frenken et al. (1992) Appl. Environ. Microb. 58: 3787-3791.) and the lipAB deficient derivative B. glumae PG1ΔlipAB (Knorr J. 2010. Physiologie eines industriellen Produktionsstammes: Proteinsekretion, Regulation and Produktion von 653 Biotensiden in Burkholderia glumae. Ph.D. thesis. Heinrich-Heine-University Duesseldorf, Duesseldorf, Germany) were cultivated in LB medium at 30° C. For analysis of lipase activities and transcript-level determination, B. glumae strains were cultivated for 14 h at 150 rpm. Standard cloning experiments were performed in E. coli DH5α. Plasmids were stabilized by using appropriate concentrations of chloramphenicol (50 μg/ml for E. coli and 200 μg/ml for B. glumae). The expression of the lipAB operon from plasmid pBBR-lipAB harboring its natural promoter was defined as native expression level.

Genome sequencing of B. glumae PG1. Genomic DNA of B. glumae PG1 was isolated with the Masterpure DNA purification Kit (Epicentre, Madison, USA) and was used to produce whole genome shotgun-libraries. For the libraries, fragments of 2.5 to 5.0 kb and 35 to 45 kb were separated by gel electrophoresis after mechanical shearing with Nebulizer devices (Invitrogen, Carlsbad, USA), end repaired and cloned in pCR2.1-TOPO (Invitrogen) for the small-insert libraries and in pCC1FOS (Epicentre) for the fosmid libraries, respectively. Plasmid and fosmid DNA were prepared using BioRobots8000 machines (Qiagen GmbH, Hilden, Germany). All inserts were automatically end-sequenced on ABI3730x1 Sequencers (Applied Biosystems, Darmstadt, Germany) using the BigDye Terminator v3.1 cycle sequencing Kit (Applied Biosystems). About 90,000 generated sequences were automatically processed with pregap and assembled into contigs with the Phrap assembly tool (http://www.phrap.org). Primer walking on plasmids, fosmid clones and PCR based techniques were used to close remaining gaps and to solve misassembled regions caused by the high number of repetitive sequences. All manual editing steps were performed using the GAP4 software package v4.6 (Staden (1996) Mol. Biotechnol. 5: 233-241.).

Genome sequencing and SNP analysis of B. glumae LU8093. The genome sequencing was carried out with a hybrid approach using the 454 GS-FLX system (Roche Life Science, Mannheim, Germany) and the Genome Analyzer IIx (Illumina, San Diego, Calif.) resulting in 437,363 454-reads and 3,998,786 solexa-reads. In order to identify SNPs, sequence reads of LU8093 were mapped against the B. glumae PG1 reference with the GS Reference Mapper (Roche Life Science, Mannheim, Germany). All candidate SNP positions were then manually revised.

Data deposition. The closed genome sequence has been deposited at the NCBI GenBank database with the Accession no. CP002580 (chromosome 1) and CP002581 (chromosome 2).

Recombinant DNA techniques. Standard DNA techniques were performed as described in Sambrook J, Fritsch E F, Maniatis T. 1989. Molecular cloning: A laboratory manual. 2nd edition. Cold Spring Harbor 673 Laboratory Press U.S. PCR Extender System (5 Prime, Hilden, Germany) was used for amplification of DNA fragments. Other DNA modifying enzymes were obtained from Thermo Scientific (St. Leon-Rot, Germany) using manufacturer's instructions. Plasmid isolation from E. coli DH5α was performed with innuPREP Plasmid Mini Kit (Analytic Jena, Jena, Germany). Genomic DNA from B. glumae PG1 (wild-type) and B. glumae LU8093 was isolated using DNeasy® Blood & Tissue Kit (Qiagen, Hilden, Germany).

The lipAB wild-type operon and the lipAB operon that harbors the mutations in the promoter region and the region coding for the LipA signal peptide were amplified using the isolated genomic DNAs from both strains as template and the primer-pair “PG1 lipAB up/dn” (ATA TAT ATC TAG AAT TCA CCG GAT CGA TCG/ATA TAT AAG CTTI ACC CGT TCG AAG CAC T). The PCR products include 249 by upstream of the startcodon of lipA with the predicted promoter sequence. The resulting DNA-fragments harboring primer introduced restriction sites were hydrolyzed with XbaIl and HindlIl and the resulting 2444 by fragments were ligated into Xbαl-HindlIl treated plasmid pBBR1-MCS (Kovach et al. (1994) Biotechniques 16: 800-802.). The resulting plasmids were named pBBR-lipAB and pBBR-lipAB-3, respectively. Plasmid pBBR-lipAB was used as template for overlap-extension-PCRs (Higuchi et al. (1988) Nucleic Acids Res 16: 7351-7367) to introduce single mutations. For the mutation in the promoter region the primer pair “OLE PCR ½” (CCT GTC TAC AAT CAG ACG GCC G/CGG CCG TCT GAT TGT AGA CAG G) was used whereas the pair “OLE PCR ¾” (GGA ACG CAT CAA TCT GAC CAT G/CAT GGT CAG ATT GAT GCG TTC C) was used for the mutation in the region coding for the signal peptide. The primer pair “PG1 lipAB up/dn” was used as flanking primers, the resulting 2463 by amplicon was then treated as described above. The resulting plasmids were named pBBR-lipΔB-1 (mutation in the promoter region) and pBBR-lipAB-2 (mutation in the signal sequence).

Transformation and conjugation. E. coli strains were transformed with plasmid DNA by heat shock transformation (Hanahan (1983) J. Mol. Biol. 166: 557-580). B. glumae strains were transformed by biparental mating with E. coli S17-1 as follows: For conjugation, 1 ml overnight culture of B. glumae was mixed with 2 ml of E. coli S17-1 in the exponential growth phase (O.D. 580 nm=0.6-0.8) containing the plasmid of interest. After centrifugation (1 min, 21,000×g), the cell pellet was washed with 0.5 ml LB medium, resuspended in 50 μl LB medium and dropped onto a membrane filter (M24, Whatman) placed on an LB agar-plate. The filter was washed off with LB medium after 6 hours at 30° C. and the cell suspension was plated in appropriate dilutions on MME (Vogel and Bonner (1956) J. Biol. Chem. 218: 97-106) agar plates containing antibiotics and 0.5% (w/v) glucose.

Western Blot analysis. Proteins from cell-free supernatants were precipitated with sodium deoxycholate and trichloroacetic acid (TCA) as described in Peterson (1977) Anal. Biochem. 83: 346-356. After washing with ½ volume 80% (v/v) acetone, the pellet was suspended with 2× SDS-sample puffer (50 mM Tris-HCl, 4% (w/v) SDS, 10% (v/v) glycerol, 10% (v/v) 2-mercaptoethanol, 0.03% (w/v) bromphenol blue).

Proteins were separated by SDS-PAGE with a 12% polyacrylamide gel (Laemmli (1970) Nature 227: 680-685.). Western blot analysis of LipA and LipB was performed using specific antibodies (kindly provided by Jan Tommassen, University of Utrecht, The Netherlands). A goat-anti-rabbit IgG (H+L)-HRP conjugate (BioRad, Munich, Germany) was used as secondary antibody. Specific antibody-protein interactions were detected using the ECL Western Blotting Detection system (Amersham Pharmacia, Buckinghamshire, GB) and the luminescence detector Stella (raytest, Straubenhardt, Germany).

Lipase assay. Lipase activity in whole cell extracts and supernatants was measured with para-nitrophenyl palmitate (mNPP) as the substrate (Winkler and Stuckmann (1979) J. Bacteriol. 138: 663-670) at 410 nm in microtiter plates using a SpectraMax 250 photometer (Molecular Devices, Ismaning/München, Germany). Relative lipase activity was correlated to cell density (OD 580 nm) and calculated as U/ml, with one U (unit) defined as the amount of lipase that releases 1 mmol of para-nitrophenol per minute (molar absorption coefficient 15 μMol⁻¹×cm⁻¹).

Transcript level determination. 2 ml of culture were centrifuged (1 min, 21,000×g) and washed once with TE buffer (100 mM Tris-HCl pH 7.5, 20 mM EDTA). The cell pellet was then treated with RNeasy Mini Kit (Qiagen) according to the protocol for the isolation of bacterial RNA. DNaseI digestion was performed both, “on column” with RNase-free DNase Set (Qiagen) and after RNA elution with DNaseI (RNase-free) from Ambion® (Life Technologies, Darmstadt, Germany) according to manufacturer's instructions.

The transcription of isolated RNA into cDNA was carried out with the High Capacity cDNA Reverse Transcription Kit (Applied Biosystems™, Foster City, USA) according to the instruction manual. For subsequent real time qPCRs, 250 ng RNA were transcribed per reaction. In a separate reaction, each sample was also treated without reverse transcription to exclude DNA contaminations.

The analysis of transcriptional levels of lipA and lipB was performed with real time qPCR (35 cycles) using the AACT-method (Livak and Schmittgen (2001) Methods 25: 402-408; Schmittgen and Livak (2008) Nat. Protoc. 3: 1101-1108). Here, the reverse transcribed cDNA was used as template in a real time 7900HT Fast Real-Time PCR System with Power SYBR® Green PCR Master Mix (both Applied Biosystems™ ), and specific primers for lipA (CTA TCC GGT GAT CCT CGT C/GAG AGA TTC GCG ACG TAC AC), lipB (GTG GCA GAC GCG CTA TCA AG/CGT GAA AGT CTG CTG CCT GAG) and the constitutively expressed gene rpoD (GAT GAC GAC GCA ACC CAG AG/GAA CGC TTC CTT CAG CAG CA) as a reference. Primers were designed using Primer3 (Untergasser et al. (2012) Nucl. Acids Res. 40(15)). The amount of PCR product was calculated as CT value by the Sequence Detection System (Version 2.3, Applied Biosystems™ ). PCR efficiencies were determined with the tool LinRegPCR (Ruijter et al. (2009) Nucl. Acids Res. 37: e45.). The CT values obtained for lipA and lipB were then referred to the reference gene rpoD leading to the ΔCT value (ΔCT=CT(gene)−CT(rpoD)). By comparing the ΔCT values of a certain strain to its reference strain, the resulting ΔΔC (ΔΔCT=CT(strain)−ΔCT(reference strain)) value reflects the differences in the transcript amount of a certain gene between these two strains. Calculations were performed and statistically analyzed with REST© software (Pfaffl et al. (2002) Nucl. Acids Res. 30:e36). All observed transcript exchanges are significantly different from the control sample (p<0.05, calculated with REST©).

### Results

Comparison of B. glumae wild-type and the lipase production strain LU8093

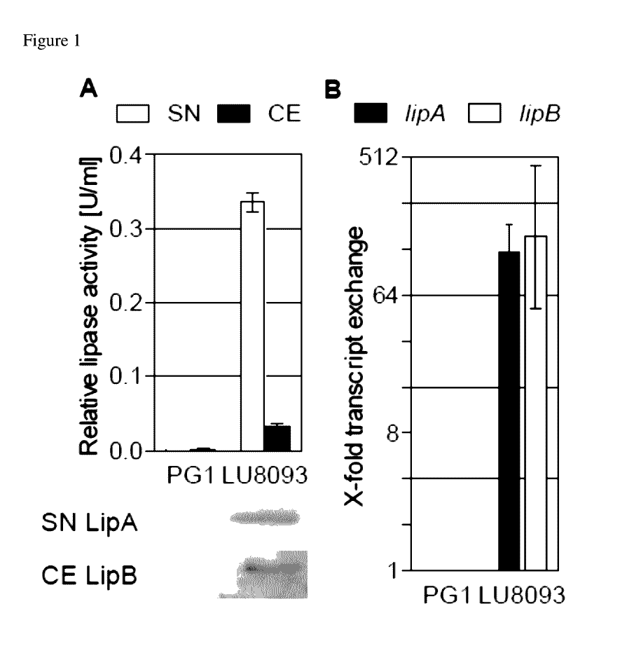

The B. glumae strain LU8093 was constructed by repeated rounds of random mutagenesis and subsequent selection for increased extracellular lipase production. The production and secretion of LipA by B. glumae LU8093 is shown in FIG. 1A. The higher production level corresponds to a 100-fold increased transcription level of the lipA gene (FIG. 1B) which is located in an operon together with a second gene lipB (or lif) encoding a lipase specific foldase. LipA possesses an N-terminal signal peptide that mediates transport through the inner membrane via the Sec secretion system. In the periplasm, the steric chaperone LipB interacts with the lipase resulting in the conversion of the enzymatically inactive so-called “near-native” state into an active conformation. Secretion through the outer membrane is subsequently achieved via the type II secretion system formed by the so-called secreton (or “main terminal branch” of the general secretory pathway).

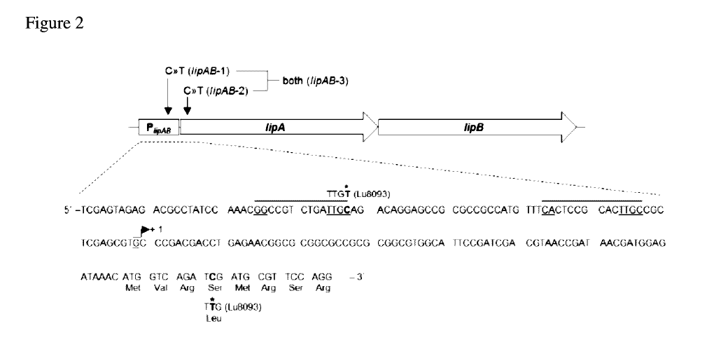

A comparison of the genome sequences of B. glumae PG1 wild-type and the production strain B. glumae LU8093 identified 72 SNPs of which two were localized within the lipase operon on chromosome 2; one in the putative promoter region and the second in the region encoding the LipA signal peptide (FIG. 2). The promoter mutation changes the δ54 consensus motif GG-N8-TTGC (Barrios et al. (1999) Nucl. Acids Res. 27: 4305-4313) from -TTGC to -TTGT (see FIG. 2). One would expect that this C to T transition decreases the lipA transcription rate, but surprisingly, it causes an increase in lipA transcript level. The second mutation identified in the lipΔB operon results in an exchange of serine to leucine at position 4 of the LipA signal peptide. The replacement of a polar serine by a hydrophobic leucine residue increases the hydrophobicity of the LipA signal peptide and may thus facilitate its interaction with the Sec-machinery thereby accelerating transport of LipA through the bacterial inner membrane (Driessen and Nouwen (2008) Annu. Rev. Biochem. 77: 643-667).

Role of two mutations localized within the lipase operon lipAB for lipase production

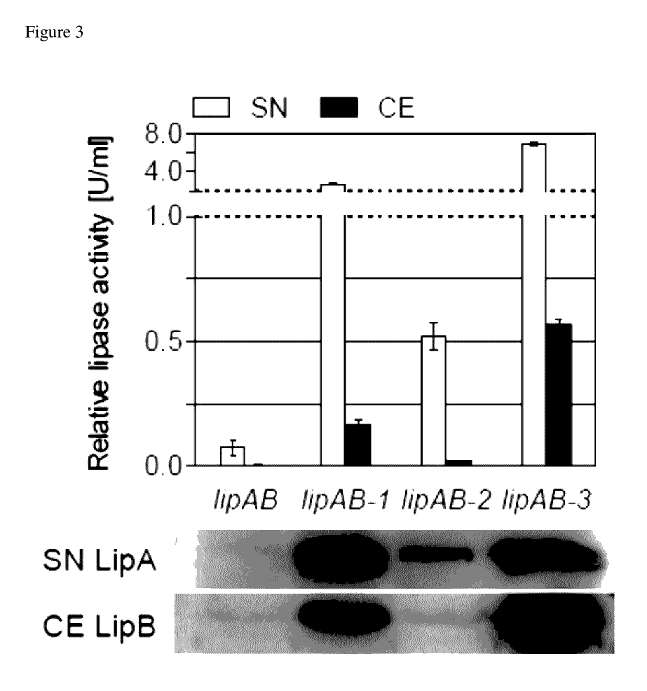

The effect of the two mutations was analyzed both separately and in combination using plasmids harboring the wild-type lipAB operon or the operon carrying the respective mutations, both expressed in a lipAB-deficient B. glumae PG1 strain (PG1ΔlipAB) to avoid basal expression of genome-encoded lipAB. To ensure that extracellular lipase activities were not caused by cell lysis, cytoplasmic β-lactamase activities were determined in cell-free culture supernatants. These activities were always less than 10% of the overall activities for all strains tested indicating that the observed effects of the mutations on extracellular lipase levels were not caused by significant cell lysis. As shown in FIG. 3 , the mutation in the promoter region of lipAB (lipAB-1) resulted in a 38-fold increased lipase activity in the supernatant (˜2.68 compared to ˜0.07 U/ml) and 42-fold in the cell extract (˜0.168 compared to ˜0.004 U/ml). The mutation in the signal peptide (lipAB-2) led to a ˜4-7-fold increase of lipase activity in the supernatant and the cell extract, whereas the combination of both mutations (lipAB-3) resulted in ˜100-fold increased activity in the supernatant (˜6.87 U/ml) and ˜140-fold increased activity (˜0.57 U/ml) in the whole cell extract. It should be noted here that lower lipase activities of B. glumae PG1 wild-type and B. glumae LU8093 as shown in FIG. 1A can be attributed to the fact that these strains harbor just one chromosomal copy of the lipAB operon. The increased lipolytic activity of B. glumae PG1ΔlipAB expressing plasmid-encoded lipase variants indeed corresponded to increased production and secretion as determined by Western blot analysis of LipA in cell-free supernatants (FIG. 3 , bottom).

1. An isolated nucleic acid molecule comprising a nucleotide sequence that is at least 80% identical to SEQ ID NO: 1 and encoding a polypeptide having a hydrophobic amino acid at a position corresponding to position 4 of the amino acid sequence as depicted in SEQ ID NO: 2.

2. The isolated nucleic acid molecule of claim 1, wherein the hydrophobic amino acid is selected from the group consisting of leucine, valine, isoleucine, methionine and alanine.

3. The isolated nucleic acid molecule of claim 1 comprising the nucleotide sequence according to SEQ ID NO: 8.

4. An isolated nucleic acid molecule comprising a nucleotide sequence that is at least 80% identical to SEQ ID NO: 3 and contains at a position corresponding to position 116 of the nucleotide sequence as depicted in SEQ ID NO: 3 a thymidine residue.

5. The isolated nucleic acid molecule of claim 4, comprising the nucleotide sequence according to SEQ ID NO: 10.

6. An isolated nucleic acid molecule comprising a first and a second nucleotide sequence,
wherein the first nucleotide sequence is at least 80% identical to SEQ ID NO: 3 and contains at a position corresponding to position 116 of the nucleotide sequence as depicted in SEQ ID NO: 3 a thymidine residue; and
wherein the second nucleotide sequence is at least 80% identical to SEQ ID NO: 1 and encodes a polypeptide having a hydrophobic amino acid at a position corresponding to position 4 of the amino acid sequence as depicted in SEQ ID NO: 2, wherein the second nucleotide sequence is located at the 3′ end of the first nucleotide sequence and is operably linked thereto.

7. The isolated nucleic acid molecule of claim 6, wherein the hydrophobic amino acid is selected from the group consisting of leucine, valine, isoleucine, methionine and alanine.

8. The isolated nucleic acid molecule of claim 6 comprising a nucleotide sequence as depicted in SEQ ID NO:4.

9. The isolated nucleic acid molecule of claim 8, further comprising a third nucleotide sequence coding for an enzyme, wherein the third nucleotide sequence is fused to the 3′ end of the nucleotide sequence as depicted in SEQ ID NO: 4.

10. The isolated nucleic acid molecule of claim 9, wherein the enzyme is a lipase and has at least 70% identity to the amino acid sequence as depicted in SEQ ID NO: 6.

11. The isolated nucleic acid molecule of claim 9, further comprising a fourth nucleotide sequence coding for a chaperone, wherein the fourth nucleotide sequence is operably linked to the third nucleotide sequence.

12. The isolated nucleic acid molecule of claim 11, wherein the chaperone has at least 70% identity to the amino acid sequence as depicted in SEQ ID NO: 7.

13. The isolated nucleic acid molecule of claim 12, comprising a nucleotide sequence as depicted in SEQ ID NO: 5 or SEQ ID NO: 11.

14. A recombinant protein comprising a polypeptide sequence, wherein the polypeptide sequence is at least 90% identical to SEQ ID NO: 2 and has a hydrophobic amino acid at a position corresponding to position 4 of the amino acid sequence as depicted in SEQ ID NO: 2.

15. The recombinant protein of claim 14, wherein the hydrophobic amino acid is selected from the group consisting of leucine, valine, isoleucine, methionine and alanine.

16. An expression vector comprising the nucleic acid molecule of claim 1.

17. An expression vector comprising the nucleic acid molecule of claim 6.

18. An expression vector comprising the nucleic acid molecule of claim 13.

19. A microorganism comprising the nucleic acid molecule of claim 1.

20. A microorganism comprising the nucleic acid molecule of claim 13.

21. The microorganism of claim 19, wherein the microorganism is a bacterium selected from the group consisting of Burkholderia glumae, Burkholderia gladioli, Burkholderia mallei, Burkholderia pseudomallei, Burkholderia thailandensis, Escherichia coli, Bacillus licheniformis, Bacillus subtilis, Bacillus lentus, Bacillus amyloliquefaciens, Bacillus alcalophilus, Bacillus globigii, Bacillus gibsonii, Bacillus clausii, Bacillus halodurans and Bacillus pumilus.

22. A recombinant microorganism comprising the expression vector of claim 16.

23. A recombinant microorganism comprising the expression vector of claim 18.

24. The recombinant microorganism of claim 22, wherein the recombinant microorganism is a bacterium selected from the group consisting of Burkholderia glumae, Burkholderia gladioli, Burkholderia mallei, Burkholderia pseudomallei, Burkholderia thailandensis, Escherichia coli, Bacillus licheniformis, Bacillus subtilis, Bacillus lentus, Bacillus amyloliquefaciens, Bacillus alcalophilus, Bacillus globigii, Bacillus gibsonii, Bacillus clausii, Bacillus halodurans and Bacillus pumilus.

25. A method for producing a lipase, wherein the method comprises cultivating the microorganism of claim 20 under conditions suitable for the production of the lipase and obtaining the lipase.

26. A lipase obtainable by the method of claim 25.

27. (canceled)

28. The isolated nucleic acid molecule of claim 10, further comprising a fourth nucleotide sequence coding for a chaperone, wherein the fourth nucleotide sequence is operably linked to the third nucleotide sequence.

29. The microorganism of claim 20, wherein the microorganism is a bacterium selected from the group consisting of Burkholderia glumae, Burkholderia gladioli, Burkholderia mallei, Burkholderia pseudomallei, Burkholderia thailandensis, Escherichia coli, Bacillus licheniformis, Bacillus subtilis, Bacillus lentus, Bacillus amyloliquefaciens, Bacillus alcalophilus, Bacillus globigii, Bacillus gibsonii, Bacillus clausii, Bacillus halodurans and Bacillus pumilus.

30. The recombinant microorganism of claim 23, wherein the recombinant microorganism is a bacterium selected from the group consisting of Burkholderia glumae, Burkholderia gladioli, Burkholderia mallei, Burkholderia pseudomallei, Burkholderia thailandensis, Escherichia coli, Bacillus licheniformis, Bacillus subtilis, Bacillus lentus, Bacillus amyloliquefaciens, Bacillus alcalophilus, Bacillus globigii, Bacillus gibsonii, Bacillus clausii, Bacillus halodurans and Bacillus pumilus.
